(19) United States
(12) Patent Application Publication
(10) Pub. No.: US 2006/0235541 A1
(43) Pub. Date: Oct. 19, 2006

(54) Bearing implant

(75) Inventor: Robert A. Hodorek, Warsaw, IN (US)

Correspondence Address: ZIMMER TECHNOLOGY - REEVES, P. O. BOX 1268, ALEDO, TX, 76008, US

(73) Assignee: Zimmer Technology, Inc.

(21) Appl. No.: 11/107,765

(22) Filed: Apr. 15, 2005

(51) Int. Cl. A61F 2/30; A61F 2/28

(52) U.S. Cl. 623/23.51; 623/23.5; 623/23.41

(57) ABSTRACT

An implant is provided having a bearing surface joined to a substrate.

### FIELD OF THE INVENTION

The invention relates to implants for skeletal joints. In particular, the invention relates to such implants having a bearing surface joined to a substrate.

### BACKGROUND

Degenerative and traumatic damage to the articular cartilage of skeletal joints can result in pain and restricted motion. Prosthetic joint replacement surgery is frequently utilized to alleviate the pain and restore joint function. During this surgery, one or more of the articulating surfaces of the joint are replaced with prosthetic bearing components. The replacement components typically include a portion for anchoring the implant adjacent to the joint and a portion for articulating with opposing joint surfaces. It is desirable for the implant to be well anchored and present a low friction, low wear articular surface.

### SUMMARY

The present invention provides a bearing implant for a skeletal joint.

In one aspect of the invention, a bearing implant for replacing a portion of an articular joint surface includes a substrate including a plurality of discrete segments and a bearing surface attached to the substrate.

In another aspect of the invention, a method of repairing an articular surface of a skeletal joint includes providing a bearing implant including a substrate including a plurality of discrete segments, the segments being separated by parting lines and a bearing surface attached to the substrate, and intraoperatively shaping the implant along one or more of the parting lines to fit a surgical site.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a top plan view of an illustrative implant according to the present invention;

FIG. 2 is a side elevation view of the implant of FIG. 1 in an unflexed condition;

FIG. 3 is a bottom plan view of the implant of FIG. 1; and

FIG. 4 is a side elevation view of the implant of FIG. 1 in a flexed condition.

### DESCRIPTION OF THE ILLUSTRATIVE EXAMPLES

Embodiments of a bearing implant include a bearing surface mounted to a substrate. The bearing implant may function as a replacement for damaged or diseased cartilage of a skeletal joint to sustain continued joint function. The bearing implant may be used to replace a portion of any skeletal joint including, but not limited to, joints of the hip, knee, shoulder, spine, elbow, wrist, ankle, jaw, and digits. The implant may be configured to replace a relatively small defect within the joint, an entire compartment of the joint, and/or the total joint.

The bearing surface may be made of any material suitable for articulation with natural or prosthetic opposing bearing surfaces. Preferably the bearing material is resilient to facilitate intraoperative flexing, cutting, and/or otherwise shaping of the bearing surface to fit a surgical site. The bearing surface may include polyolefins, polyesters, polyimides, polyamides, polyacrylates, polyketones, and/or other suitable materials. For example the bearing surface may include ultrahigh molecular weight polyethylene.

The bearing surface may include a hydrogel having a three dimensional network of polymer chains with water filling the void space between the macromolecules. The hydrogel may include a water soluble polymer that is crosslinked to prevent its dissolution in water. The water content of the hydrogel may range from 20-80%. The high water content of the hydrogel results in a low coefficient of friction for the bearing due to hydrodynamic lubrication. Advantageously, as loads increase on the bearing component, the friction coefficient decreases as water forced from the hydrogel forms a lubricating film. The hydrogel may include natural or synthetic polymers. Examples of natural polymers include polyhyaluronic acid, alginate, polypeptide, collagen, elastin, polylactic acid, polyglycolic acid, chitin, and/or other suitable natural polymers and combinations thereof. Examples of synthetic polymers include polyethylene oxide, polyethylene glycol, polyvinyl alcohol, polyacrylic acid, polyacrylamide, poly(N-vinyl-2-pyrrolidone), polyurethane, polyacrylonitrile, and/or other suitable synthetic polymers and combinations thereof.

The substrate provides support for the hydrogel and/or provides an anchor for the implant. The substrate may be solid or porous. The bearing surface may attach to the substrate by bonding, mechanical fasteners, porous interdigitation, and/or other suitable attachment methods. For example, the substrate may include an open porous structure in which a portion of the bearing surface is integrated to attach the bearing surface to the substrate. The substrate may be configured to be cemented in place, to be press-fit in place, to receive tissue ingrowth, and/or to be anchored to tissue in any other suitable tissue anchoring configuration. For example, the substrate may include an open porous structure for placement adjacent to body tissue to receive tissue ingrowth to anchor the implant adjacent the tissue. A porous structure may be configured to promote hard and/or soft tissue ingrowth. The porous structures may be in form of an open cell foam, a woven structure, a grid, agglomerated particles, and/or other suitable structures and combinations thereof.

The substrate may include any suitable material including, but not limited to, metals, polymers, ceramics, hydrogels and/or other suitable materials and combinations thereof. For example, a polymer substrate may include resorbable and/or non-resorbable polymers.

Examples of resorbable polymers include polylactic acid polymers, polyglycolic acid polymers, and/or other suitable resorbable polymers. Examples of non-resorbable polymers include polyolefins, polyesters, polyimides, polyamides, polyacrylates, polyketones, and/or other suitable non-resorbable polymers. A metal substrate may include titanium, tantalum, stainless steel, and/or other suitable metals and alloys thereof. Preferably the substrate is relatively rigid to provide a suitable surface for hard tissue ingrowth. For example, the substrate may include a porous tantalum material having a structure similar to that of natural trabecular bone. Such a material is described in U.S. Pat. No. 5,282,861 entitled “OPEN CELL TANTALUM STRUCTURES FOR CANCELLOUS BONE IMPLANTS AND CELL AND TISSUE RECEPTORS”. The material is fabricated by vapor depositing tantalum into a porous matrix. The substrate may include protruding pegs or other bone engaging features to further enhance the connection of the substrate to tissue.

Tissue growth promoting substances may be included in the substrate and/or added at the time of surgery. Examples of tissue promoting substances include hydroxyapitite, particulate bone, bone growth proteins, autologous tissue derived growth factors, bone marrow aspirate, stem cells, and/or other tissue growth promoting substances.

The substrate may be formed into discrete segments to facilitate intraoperative flexing, cutting, tearing and/or otherwise shaping of the substrate to fit a surgical site. The segments may be formed from a continuous piece of substrate material by cutting, scoring, punching, molding, and/or otherwise forming the substrate. The segments may be completely separated or they may include some interconnecting substrate material as with a scored substrate being cut partway through between segments. The segments may be formed before or after the substrate and bearing surface are joined. For example a piece of substrate material may be joined to a bearing surface and subsequently the substrate may be cut to form discrete segments. In another example, the segments may be provided as discrete segments to which a bearing material is subsequently joined. The segments may abut one another or they may be spaced apart. The substrate material may be relatively more rigid than the bearing surface material.

The bearing surface may be formed by casting, injection molding, compression molding, machining, and/or other suitable forming processes and combinations thereof. For example, the bearing surface may be compression molded onto a porous substrate such that the bearing surface interdigitates with the substrate and is thereby joined to it.

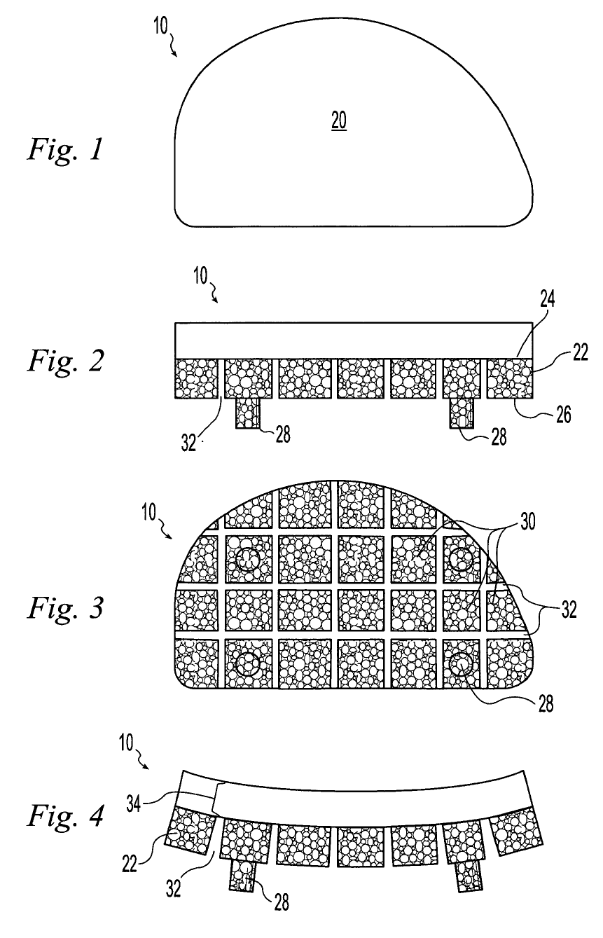

FIGS. 1-4 depict an illustrative example of a bearing implant 10 according to the present invention. The illustrative implant 10 is in the form of a unicondylar tibial knee joint prosthesis. However, it is within the scope of the invention for the bearing implant 10 to be configured to replace a small portion of the tibial articular bearing surface, to replace an entire compartment of the tibial articular bearing surface, to replace both compartments of the tibial articular bearing surface, to replace the femoral condyles of the knee joint, and/or to replace any amount of any bearing surface in any skeletal joint. The implant 10 includes a bearing surface 20 configured to receive an opposing portion of the joint in articulating relationship and a substrate 22. The substrate 22 preferably includes a first porous region 24 into which a portion of the bearing surface 20 is interdigitated to connect the bearing surface 20 to the substrate 22. In the illustrative example, a hydrogel bearing surface 20 is molded into the pores of the first porous region 24. Preferably the substrate 22 includes a second porous region 26 for placement against tissue for receiving tissue ingrowth. In the illustrative example, the substrate 22 is porous tantalum and is porous throughout to provide first and second porous regions 24 and 26. The illustrative substrate 22 includes protruding pegs 28 for insertion into holes formed in an underlying bone to further enhance the connection of the substrate 22 to the bone.

In the illustrative example, the substrate 22 is formed into a grid of discrete, generally planar segments 30 separated by parting lines 32. The parting lines 32 facilitate intraoperative flexing, tearing, cutting, and/or otherwise shaping the implant 10. For example, the parting lines 32 result in a thinner region 34 along which the implant 10 is more flexible. The parting lines 32 may be relatively narrow (not shown) so that the segments 30 abut one another in an unflexed state and appear as one continuous substrate surface. In this configuration, the implant 10 will be more flexible in a direction that tends to open the parting lines 32 and be more rigid in a direction that tends to press the segments 30 together. Alternately, the parting lines 32 may be relatively wide (as shown) to provide a gap between segments 30 to facilitate flexing of the implant 10 both in directions that tend to open the parting lines 32 (FIG. 4) and in directions that tend to close the parting lines 32. The parting lines may extend all the way through the substrate 22 (as shown) or they may be scored only partway through the substrate 22. The number and shape of the segments 30 and parting lines 32 may be tailored for particular applications to enhance and/or restrict flexibility in portions of the implant 10. For example, the implant may have two segments 30 separated by a single parting line 32 allowing the two segments to flex relative to one another along the single parting line. The implant 10 may have any number of segments 30 suitable to a particular application. In the illustrative example, the bearing surface 20 provides a relatively flexible, lubricious bearing surface 20, while the segments 30 provides individual, relatively rigid bone mounting surfaces.

The parting lines 32 also facilitate cutting, tearing and/or otherwise shaping the substrate 22. The parting lines 32 present thinner regions 34 of the implant that may be more easily cut with a knife, scissors, shears, or other cutting instrument. The parting lines 32 may extend all the way through a difficult to cut substrate 22, such as a metal substrate 22 (as shown), so that only the bearing surface 20 need be cut intraoperatively. With some materials, the parting lines 32 may make it possible to tear away unneeded segments. The number and shape of the segments 30 and parting lines 32 may be tailored to define predetermined implant shapes corresponding to different surgical sites, differing patient anatomy, and/or different defect shapes and/or sizes. The user can selectively shape the implant along a desired parting line to match the implant shape to the particular use.

In use, the implant 10 is compared to a cartilage region that is to be repaired. The shape of the desired replacement is marked on the implant 10 and then the implant is flexed, torn, cut and/or otherwise reshaped along the parting lines 32 to approximate the desired replacement. The implant 10 is then anchored to the underlying tissue by cementing, press fitting, and/or juxtaposing it for hard and/or soft tissue ingrowth. In the illustrative example, holes are drilled into underlying bony tissues and the pegs 28 are pressed into the holes with the segments 30 abutting the underlying bony tissues to facilitate bony ingrowth into the pegs 28 and segments 30 to anchor the implant 10.

Although examples of a bearing implant and its use have been described and illustrated in detail, it is to be understood that the same is intended by way of illustration and example only and is not to be taken by way of limitation. The invention has been illustrated in the context of a tibial articular implant. However, the bearing implant may be configured in other shapes and for use at other locations within a patient's body. Accordingly, variations in and modifications to the bearing implant and its use will be apparent to those of ordinary skill in the art, and the following claims are intended to cover all such modifications and equivalents.

What is claimed is:

1. A bearing implant for replacing a portion of an articular joint surface, the implant comprising:
a substrate comprising a plurality of discrete segments; and
a bearing surface attached to the substrate.

2. The bearing implant of claim 1 wherein the bearing surface comprises a continuous smooth surface for articulation with the articular joint.

3. The bearing implant of claim 2 wherein the bearing surface comprises a polymer.

4. The bearing implant of claim 3 wherein the polymer comprises a hydrogel.

5. The bearing implant of claim 3 wherein the polymer comprises ultrahigh molecular weight polyethylene.

6. The bearing implant of claim 3 wherein the substrate comprises a metal layer.

7. The bearing implant of claim 6 wherein the substrate comprises a first porous region in which a portion of the bearing surface interdigitates to join the bearing surface to the substrate.

8. The bearing implant of claim 7 wherein the substrate comprises a second porous region for receiving tissue ingrowth to anchor the implant adjacent the joint.

9. The bearing implant of claim 8 wherein the substrate comprises an open cell porous tantalum material.

10. The bearing implant of claim 1 wherein the substrate material is relatively more rigid than the bearing component material such that the bearing surface is relatively flexible and the segments are individually relatively more rigid.

11. The bearing implant of claim 1 wherein the segments are separated by parting lines.

12. The bearing implant of claim 11 wherein the implant is shapeable by cutting along the parting lines.

13. The bearing implant of claim 11 wherein the implant is shapeable by tearing along the parting lines.

14. The bearing implant of claim 11 wherein the parting lines are formed partway through the substrate.

15. The bearing implant of claim 11 wherein the parting lines are formed completely through the substrate.

16. The bearing implant of claim 1 wherein the implant is relatively flexible between segments such that the implant may be flexed by moving segments relative to one another.

17. The bearing implant of claim 16 wherein the implant has an unflexed condition in which adjacent segments abut one another and a flexed condition in which adjacent segments are spaced apart.

18. The bearing implant of claim 17 wherein the implant is relatively less flexible in a direction that presses the segments together than in a direction the moves the segments apart.

19. The bearing implant of claim 16 wherein the implant has an unflexed condition in which adjacent segments are spaced apart with an intervening gap between segments, the implant being relatively flexible both in directions that tend to move the segments further apart and in directions that tend to move the segments closer together.

20. The bearing implant of claim 1 wherein the substrate further comprises at least one fixation peg projecting outwardly from the substrate.

21. The bearing implant of claim 1 further comprising a tissue growth promoting substance incorporated into the substrate.

22. A method of repairing an articular surface of a skeletal joint, the method comprising:
providing a bearing implant having a substrate comprising a plurality of discrete segments, the segments being separated by parting lines; and a bearing surface attached to the substrate;
intraoperatively shaping the implant along one or more of the parting lines to fit a surgical site.

23. The method of claim 22 wherein shaping the implant comprises flexing the implant.

24. The method of claim 22 wherein shaping the implant comprises cutting the implant along one or more of the parting lines.

25. The method of claim 22 wherein shaping the implant comprises tearing the implant along one or more of the parting lines.
